(19) United States
(12) Patent Application Publication
(10) Pub. No.: US 2006/0057647 A1
(43) Pub. Date: Mar. 16, 2006

(54) Detection and identification of saxiphilins using saxitoxin-biotin conjugates

(76) Inventor: Cedic Emile Francois Robillot, Chuwar (AU)

Correspondence Address: MOORE & VAN ALLEN PLLC, P.O. BOX 13706, Research Triangle Park, NC, 27709, US

(21) Appl. No.: 10/545,214

(22) PCT Filed: Feb. 12, 2004
(86) PCT No.: PCT/AU04/00166

§ 371 Date: Aug. 10, 2005

(30) Foreign Application Priority Data
Feb. 12, 2003 (AU) 2003901897

(51) Int. Cl. G01N 33/53

(52) U.S. Cl. 435/7.5

(57) ABSTRACT

A method for capturing a saxiphilin to allow for detection, characterisation, isolation an/or purification of said saxiphilin or its ligand, comprising: (1) providing a PST conjugate comprising a PST moiety bound via a linker through a site other than the binding site for saxiphilin directly or indirectly to a biotin moiety; (2) exposing the PST conjugate to a sample putatively containing said saxiphilin to create a reaction mixture and to (strept)avidin; and (3) allowing binding through the PST moiety to the saxiphilin and through the biotin moiety to (strept)avidin to form a captured PST complex.

### TECHNICAL FIELD

The present invention is concerned with a paralytic shellfish toxin conjugate. In particular, it is concerned with use of the paralytic shellfish toxin conjugate in the detection, characterisation, isolation and/or purification of molecules of interest, particularly the saxiphilins and their ligands, although its use is not so-limited.

### BACKGROUND ART

The so-called “saxiphilins” are a diverse class of polypeptides characterised through their ability to bind saxitoxin, one of the paralytic shellfish toxins (or PSTs). The term “saxiphilin” is a coined term including the prefix “saxi” from saxitoxin and the suffix “philic” which denotes a likening for saxitoxin. The saxiphilins do not share any particular chemical structure or physiological function, nor would it seem that the physiological purpose of the saxiphilins is necessarily to bind saxitoxin. For example, so-called “bullfrog saxiphilin” is a molecule which shares over 50% amino acid sequence identity with the transferrin class of iron-binding proteins, and so is also presumed to be a transferrin. The sodium channel also binds saxitoxin and could therefore be described as a “saxiphilin”. So-called saxiphilins have been isolated from diverse sources such as the blood of the puffer fish. This protein, like the sodium channel, binds both saxitoxin and tetrodotoxin but, unlike the sodium channel, the puffer fish protein is hydrophilic.

The sodium channel, the puffer fish saxiphilin and the transferring are each members of distinct classes of the saxiphilins. There is no amino acid sequence homology discernible between these three classes of saxiphilins. However, they may be delineated on the basis of their physical properties. The sodium channel is hydrophobic as it is anchored in a lipid membrane, whereas the other classes are hydrophilic. The transferrin class of saxiphilin binds saxitoxin but not tetrodotoxin, whereas the sodium channel and puffer fish saxiphilin bind both saxitoxin and tetrodotoxin. A further property which may be used to distinguish these molecules is the ability to bind neosaxitoxin (neoSTX). In addition, there are a great many related toxins, known as paralytic shellfish toxins, similar in structure to saxitoxin to which such molecules bind to differing extents.

Paralytic shellfish poisoning caused by ingestion of fish, crustaceans or molluscs containing toxins derived from dinoflagellates is a world-wide problem resulting in severe human illness, which often results in death. The poisoning is caused by the paralytic shellfish toxins (PSTs). In addition, blooms of toxic freshwater algae can contaminate water supplies with the same neurotoxins that cause paralytic shellfish poisoning. This toxin-contaminated water can have dire consequences for humans, livestock and wildlife.

The PSTs have the following structure, as illustrated by general formula (I):

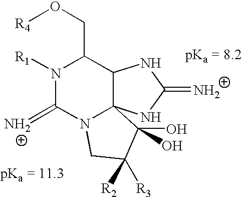

| | R₁ | R₂ | R₃ | R₄ |
|---|---|---|---|---|
| STX | H | H | H | CONH₂ |
| dcSTX | H | H | H | H |
| B1 | H | H | H | CONHSO₃⁻ |
| B2 | OH | H | H | CONHSO₃⁻ |
| C1 | H | H | OSO₃⁻ | CONHSO₃⁻ |
| C2 | H | OSO₃⁻ | H | CONHSO₃⁻ |
| C3 | OH | H | OSO₃⁻ | CONHSO₃⁻ |
| C4 | OH | OSO₃⁻ | H | CONHSO₃⁻ |
| neoSTX | OH | H | H | CONH₂ |
| dcNeoSTX | OH | H | H | H |
| GTX2 | H | H | OSO₃⁻ | CONH₂ |
| GTX3 | H | OSO₃⁻ | H | CONH₂ |
| GTX1 | OH | H | H | CONH₂ |
| GTX4 | OH | OSO₃⁻ | H | CONH₂ |
| GC1 | H | H | H | CO—C₆H₅—OH |
| GC2α | H | H | OSO₃⁻ | CO—C₆H₅—OH |
| GC2β | H | OSO₃⁻ | H | CO—C₆H₅—OH |
| GC3 | OH | H | H | CO—C₆H₅—OH |

This family of toxins can be divided into four broad categories: the saxitoxins, which are highly potent neurotoxins, and which are not sulfated; the gonyautoxins (GTXs), which are singly sulfated; the N-sulfocarbamoyl-13-hydrosulfate C-toxins, which are less toxic than the STXs or GTXs and the GC toxins which carry a phenolic group on C13.

The toxicity of the PSTs is a result of their binding to voltage-dependent sodium channels, which blocks the influx of sodium ions, and thus blocks neuromuscular transmission. This causes respiratory paralysis, for which no treatment is available. In some outbreaks of paralytic shellfish poisoning up to 40% of the victims have died. The dinoflagellates which are the source of PSTs periodically form algal blooms, known as red tides. Molluscs, fish, and crustaceans, including species of commercial significance or which are raised using aquaculture techniques, may feed on these dinoflagellates and accumulate the toxins. It is not possible to detect by gross examination whether an individual marine animal contains the toxin, and therefore there is a risk that humans will inadvertently consume toxin-containing animals. It is therefore necessary to monitor species which are to be consumed for the presence of PSTs, in order to avoid the risk of poisoning and to prevent social and economic cost.

An improved assay for saxitoxins is disclosed in our co-pending International Application No. WO02/48671. The international application discloses a method of detecting and/or measuring the amount of a paralytic shellfish toxin present in a sample by way of its binding to an isolated and purified saxiphilin molecule such as the saxiphilin isolated from the centipede Ethmostigmus rubripes. It would be desirable to have available saxiphilins from other sources which exhibit equal or better binding properties to the centipede saxiphilin. However, the saxiphilins have proven to be a difficult group of compounds to isolate and purify and, to date, only bullfrog saxiphilin is well characterised. It would also be desirable to be able to label PSTs or immobilise them on a solid support for the detection and characterisation of saxiphilin or its ligand.

### SUMMARY OF THE INVENTION

The present inventors have developed a technique whereby a PST such as saxitoxin is biotinylated in order that an avidin/streptavidin system may be employed to allow for detection, characterisation, isolation and/or purification of molecules of interest such as saxiphilins and their ligands. The combination of PST and biotin functionalities in the molecule enables a great many applications involving (strept)avidin/biotin binding which can be exploited in assay design in conjunction with PST/saxiphilin binding activity.

Accordingly, in one aspect of the present invention there is provided a method for capturing a saxiphilin to allow for detection, characterisation, isolation and/or purification of said saxiphilin or its ligand, comprising:
(1) providing a PST conjugate comprising a PST moiety bound via a linker through a site other than the binding site for saxiphilin to a biotin moiety;
(2) exposing the PST conjugate to a sample putatively containing said saxiphilin to create a reaction mixture and to (strept)avidin; and
(3) allowing binding through the PST moiety to the saxiphilin and through the biotin moiety to (strept)avidin to form a captured PST complex.

In a further aspect of the invention there is provided a method for the detection, characterisation, isolation and/or purification of a saxiphilin, comprising:
(1) providing a PST conjugate comprising a PST moiety bound via a linker through a site other than the binding site for saxiphilin to a biotin moiety;
(2) exposing the PST conjugate to a sample putatively containing the saxiphilin to create a reaction mixture and to (strept)avidin; and
(3) allowing binding through the PST moiety to the saxiphilin and through the biotin moiety to (strept)avidin to form a captured PST complex; and
(4) effecting detection, characterisation, isolatin and/or purification of the saxiphilin through the captured PST complex.

Advantageously the biotin moiety is bound to an immobilised (strept)avidin molecule for use as a medium for affinity purification. In this embodiment the PST conjugate could be immobilised on the solid phase through binding the (strept)avidin prior to exposure and the reaction mixture created through exposure of the immobilised PST conjugate to the sample. Alternatively, the PST conjugate could be exposed to the sample to form the reaction mixture while in solution, and the reaction mixture exposed to an immobilised streptavidin, for example, on an affinity column or beads, in order to capture the PST complex already formed in solution.

Any suitable affinity matrix may be used. A suitable affinity gel should have a high porosity to allow maximum access of macromolecules to the immobilised ligand, it should be of uniform size and rigidity to allow for good flow characteristics, and mechanically and chemically stable. Typical insoluble support materials include cellulose, polystyrene gels, cross-linked dextrans, polyacrylamide gels, porous silicas and agarose and derivatives thereof such as Sepharose. Column preparation can be performed using standard techniques as would be understood by the person skilled in the art. Elution of the bound saxiphilin may be achieved through changing conditions such as buffer pH, ionic strength or temperature so that the affinity of the matrix for the bound saxiphilin is reduced and/or through the addition of a competing ligand to the elution buffer, as would be well understood by the person skilled in the art. Any suitable bead support including magnetic beads or dendrimer support may also be used in a similar-approach. Advantageously the biotin moiety is bound to an immobilised (strept)avidin molecule for use as a capture probe in a PST biosensing device. In this embodiment the PST conjugate could be immobilised on the solid phase through binding the (strept)avidin prior to exposure, and the reaction mixture created through exposure of the immobilised PST conjugate to the sample containing both known amounts of saxiphilin and PST. Alternatively, the PST conjugate could be exposed to the sample to form the reaction mixture while in solution, and the reaction mixture exposed to an immobilised streptavidin, for example, on a membrane, a microlever, an electrode, or a chemically activated glass surface, in order to capture the PST complex already formed in solution. The amount of PST-saxiphilin complex adsorbed on the solid phase through binding the (strept) avidin would then be correlated to the amount of PST in the sample. Typical platforms include electrochemical, optical, surface plasmon resonance, acoustic wave, microcantilever and ion-channel switching biosensors.

In an embodiment the PST conjugate of the invention can be used as a probe to detect the presence of saxiphilins and their ligands in tissues, cells or elsewhere. Binding saxiphilin occurs through the PST moiety and detection occurs through the biotin moiety in the conventional manner. For example, fluorescent, radioactive or chemiluminescent conjugates of (strept) avidin may be used. Other detectable labels which may be applied to (strept) avidins include CMNB-caged fluorescein conjugates of (strept) avidin which can be used for light-mediated tagging or fluorescence resonance energy transfer reagents, fluorescent microsphere labels, colloidal gold, latex beads, liposomes, dendrimers, oligonucleotides, peptidonucleic acids and the like. Furthermore, enzyme-linked processes may be used for detection and therefore the (strept) avidin employed may be an enzyme conjugate such as a (strept) avidin conjugate of alkaline phosphatase, horseradish peroxidase and beta-galactosidase. All anti-(strept) avidin antibodies (labelled or not) may also be used for detection.

Alternatively, saxiphilin may be bound onto a solid phase, for example, on a membrane, a microlever, an electrode or a chemically activated glass surface, and may then capture the labelled PST complex. Competition experiments may then be run as would be well understood by the person skilled in the art.

In another embodiment, saxiphilin preferably comprises a label, preferably a label suitable for detection. A label preferably is selected from the group consisting of fluorescent label, radioactive label, chemiluminescent label, colloidal gold, latex bead, liposome, dendrimer, oligonucleotide, peptidonucleic acid, protein, antibody (directly labelled and unlabelled) and enzyme. More preferably, the enzyme is selected from the group consisting of alkaline phosphatase, horseradish peroxidase and beta-galactosidase.

In addition, the conjugate may be used to screen for specific antibodies to PSTs or DNA or RNA aptamers which bind PSTs.

Accordingly, in a further aspect of the present invention there is provided a method for capturing a an antibody to a PST or a DNA or RNA aptamer which binds PSTs, comprising:
(1) providing a PST conjugate comprising a PST moiety bound via a linker through a site other than the binding site for saxiphilin to a biotin moiety;
(2) exposing the PST conjugate to a sample putatively containing said antibody or aptamer to create a reaction mixture and to (strept)avidin; and
(3) allowing binding through the PST moiety to the antibody or aptamer and through the biotin moiety to (strept)avidin to form a captured PST complex.

Advantageously the PST is one of the PSTs classified as a saxitoxin and, more particularly, is saxitoxin itself.

In a further aspect of the present invention there is provided a PST conjugate for use in a biotin/(strept)avidin system comprising a PST bound via a linker and through a site other than the binding site for saxiphilin to biotin.

Advantageously, the linker is joined to the PST through C₁₂ or C₁₃ of saxitoxin or the equivalent position in other PSTs, preferably through C₁₃. Linkage may be through any suitable linking group and may be formed through a reaction with the pre-existing functional group on the PST or by reaction with a group introduced by modification of the PST.

Any suitable means of introducing a linker of suitable length may be employed, and diverse chemistries may be employed in extending the linker.

In a particularly preferred embodiment of the invention, linkage takes place through C₁₃ of saxitoxin itself following decarbamoylation.

In one embodiment, the reaction involves formation of an ester linkage. Advantageously, dcSTX is reacted with a dicarboxylic acid (or the corresponding anhydride). In a particularly preferred form of the invention the dicarboxylic acid is succinic acid/anhydride so as to form dcSTX hemisuccinate. Advantageously the dicarboxylic acid derivative is reacted with a hydrazine derivative of biotin to link biotin to the PST.

Alternatively, dcSTX is reacted with a isocyanate which contains a functional group able to link to a biotin derivative, such as N-(p-Maleimidophenyl) isocynate which is able to react with a sulfhydryl derivative of biotin.

It will be appreciated that the linker may comprise a carbon chain that can be interrupted by functional groups and/or heteroatoms and/or cyclic structures including cycloalkyl, heterocyclic and aromatic ring structures, and is optionally substituted. In a particularly preferred embodiment of the invention the linker is greater than 4 atoms in length (or the equivalent length wherein cyclic structures are present in the linker) in order to facilitate binding of the full range of saxiphilins. However, binding affinity is significantly improved by extending the linker, and a linker 5 atoms or greater in length is preferred. While only economics and lack of a practical synthesis places an upper limit on the length of the linker, a linker 5 to 20 atoms in length is preferred. Still more preferred is a linker 8 to 18 atoms in length and most preferred is a linker 11 to 18 atoms in length. While not wishing to be bound by theory, it is believed that some of the saxiphilins undergo a conformational change on binding a PST which places steric restraints on the binding reaction, although the extent of these restraints will differ between the saxiphilins dependent on their nature.

According to a further aspect of the present invention there is provided a complex comprising a PST conjugate complexed to saxiphilin through the PST moiety.

According to a further aspect of the present invention there is provided a complex comprising a PST conjugate complexed to (strept)avidin through the biotin moiety.

According to a further aspect of the present invention there is provided an affinity purification medium comprising a PST conjugate as described above.

According to a still further aspect of the present invention there is provided a PST biosensing device comprising a PST conjugate as described above.

According to a still further aspect of the invention there is provided a PST biosensing device comprising a saxiphilin covalently linked to a solid support and means for detecting bind of a PST conjugate thereto.

Typically the PST conjugate is bound to (strept)avidin which allows binding to be detected, for example, by the change in mass upon binding.

The term “paralytic shellfish toxin” or PST as used herein refers to a compound with a general formula I as recited above, or variants thereof which are toxic to mammals as a result of their ability to bind to voltage-dependent sodium channels.

A “PST residue” or “PST moiety” as the terms are used herein refers to the residue of a PST following a linking reaction, including such reactions where a functional group is removed as a precursor (such as decarbamoylation at C₁₃ or reduction at Cl₂ to produce saxitoxinol), and so constitutes those atoms from the PST which remain in the reaction product.

As used herein the term “biotin” refers to the compound biotin itself and derivatives thereof which retain the ability to bind avidin or streptavidin, such as desthiobiotin and derivatives thereof, which are capable of reversible binding to (strept)avidin.

The term “(strept)avidin” as used herein refers to either of the polypeptides streptavidin or avidin themselves, or modified forms of streptavidin or avidin (including fragments thereof) which retain the ability to bind biotin. In particular, avidin or streptavidin that have been deglycosylated, such as NeutrAvidin biotin-binding protein (Invitrogen) and a selectively nitrated avidin derivative (CaptAvidin, Invitrogen) whose affinity is reduced sufficiently to allow reversible binding of biotin, are envisaged.

As used herein the term “biotin residue” or “biotin moiety” refers to the residue of biotin itself or derivatives thereof as encompassed by the term “biotin” as defined above following a linking reaction.

A “biotin/(strept)avidin system”, or equivalent terms, is any system, including assays, labelling reactions, purifications and syntheses, which involve a binding interaction between (strept)avidin as defined herein and biotin as defined herein, no matter what other moieties may be involved. As an alternative to (strept)avidin, an anti-biotin antibody may be employed for detection of biotin.

As used herein the term “saxiphilin” refers to any member of a class of proteins with diverse functions characterised by their ability to bind saxitoxin. The saxiphilins include transferring with this property, the sodium channel and other hydrophobic or membrane-bound proteins with this property and a group of hydrophilic proteins which bind both saxitoxin and tetrodotoxin such as pufferfish saxiphilin, irrespective of their source, chemical nature or structure provided that the protein is functional in its usual physiological role, be that known or unknown. Given that aspects of the invention are concerned with the detection, isolation and characterisation of previously unknown saxiphilins it will be appreciated that both known and previously undiscovered molecules with this property are envisaged through use of the term.

Throughout this specification and the claims, the words “comprise”, “comprises” and “comprising” are used in a non-exclusive sense, except where the context requires otherwise.

It will be clearly understood that, although a number of prior art publications are referred to herein, this reference does not constitute an admission that any of these documents forms part of the common general knowledge in the art, in Australia or in any other country.

### BRIEF DESCRIPTION OF THE DRAWINGS

FIG. 1 is a mass spectrum of the saxitoxin conjugate Biotin-link11-STX prepared in Example 1.

FIG. 2 is a mass spectrum of the saxitoxin conjugate Biotin-link4-STX prepared in Example 2.

FIG. 3 shows data in graph form illustrating the effective competition between compounds synthesized in Example 1 and radioactive saxitoxin in saxiphilin receptor binding assays, indicating that the compound of the invention binds saxiphilin.

FIG. 4 shows a graph illustrating the effective competition between complexes synthesized in Example 2 and radioactive saxitoxin in saxiphilin receptor binding assays, indicating that the complexes of the invention bind saxiphilin and that the length of the linker is influencing their affinity for saxiphilin.

FIG. 5 shows the detection of standard saxitoxin by surface plasmon resonance using Biotin-link11-STX immobilised onto a streptavidin coated membrane as capture probe for saxiphilin.

### MODES FOR PERFORMING THE INVENTION

### EXAMPLE 1

Synthesis of Biotin-link11-STX and Synthesis of Biotin-Link 4-STX

Saxitoxin isolated from shellfish was converted to decarbamoyl-saxitoxin (dcSTX) by hydrolysis in HCl 6M in a sealed, evacuated glass tube at 110° C. for 4 hours. The solution was freeze dried. The residue was redissolved in 0.05M acetic acid and the solution passed through a C18 solid phase extraction cartridge. dcSTX was purified by Biogel-P2 chromatography and freeze-dried.

dcSTX was then redissolved in sodium phosphate buffer 0.1M pH 6.8 and converted to dcSTX-hemisuccinate by reaction with two successive additions of Succinic anhydride (ratio dcSTX:succinic anhydride 1:20) for 2 hours at 10° C. while maintaining the temperature at 10° C. and the pH at 5.7±0.1. dcSTX hemisuccinate was then separated from dcSTX and purified by anion exchange chromatography using sodium phosphate buffer 0.01 M as eluting solvent, and by Carbograph graphitized carbon solid phase extraction using ultrapure water as eluting solvent.

dcSTX hemisuccinate was then freeze-dried thoroughly and reacted overnight at room temperature with 4 moles equivalent of either Biotin-hydrazide or Biotin-LC-hydrazide in presence of 4 moles equivalent HATU (O-(7-Azabenzotriazol-1-yl)-N,N,N′,N′-tetramethyluronium hexafluorophosphate) to produce Biotin-link4-STX and Biotin-link11-STX, respectively.

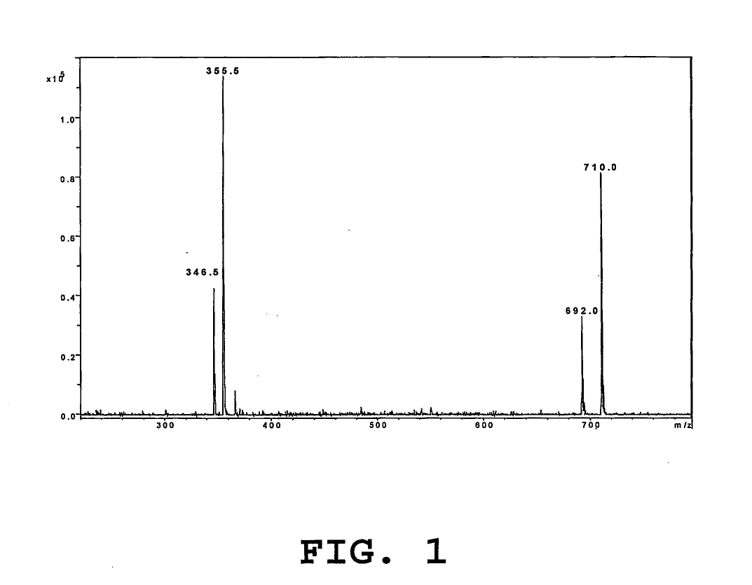

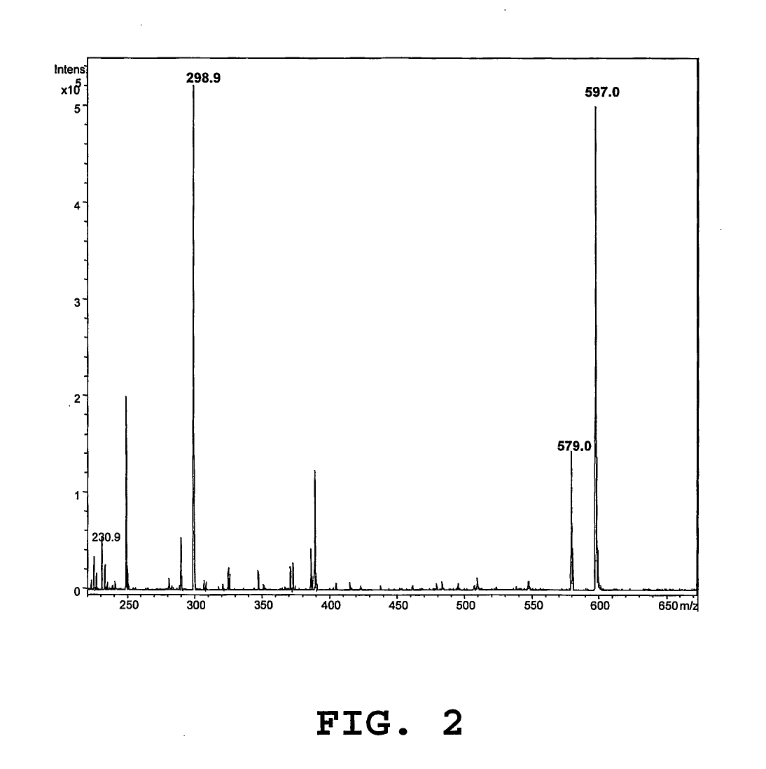

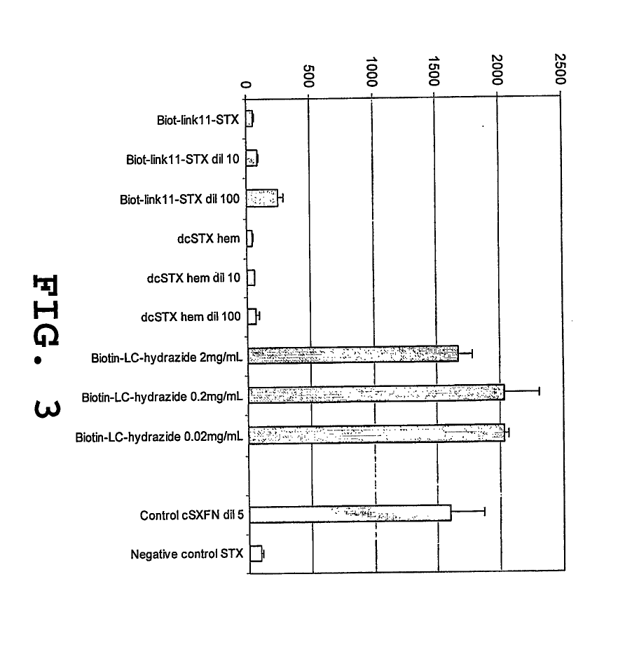

Biotin-link4-STX and Biotin-link11-STX were then characterised and purified by hydrophilic interaction chromatography—Mass spectrometry (HIC LC-MS) using an Agilent 1100 Series LC coupled to an Esquire 3000+ quadrupole ion-trap mass spectrometer (Bruker Daltonics, Billerica Mass., USA) fitted with an electrospray ionisation interface (HV capillary +4 kV, skimmer voltage 40 V). LC separation was achieved on a TSK-Gel Amide-80 column (5 μm, 250 mm×4.6 mm i.d.; TosoHass) maintained at 40° C. with an isocratic solution of 2 mM ammonium formate, 3.6 mM formic acid in 50% acetonitrile:water at 1 mL min⁻¹. The purified compounds were freeze-dried and stored for further testing. The mass spectrum of Biotin-link11-STX (FIG. 1) evidenced the presence of a singly-charged ion at m/z=710.0±0.5 Da as well as a doubly-charged ion at m/z=355.5±0.5 Da. The mass spectrum of Biotin-link4-STX (FIG. 2) evidenced the presence of a singly-charged ion at m/z=597.0±0.5 Da as well as a doubly-charged ion at m/z=298.9±0.5 Da.
The data illustrated in FIG. 3 confirms that biotin-link11-STX binds saxiphilin. The receptor binding assay employed is described in the co-pending International Application No. WO02/48671, the contents of which are incorporated herein by reference. Filtering 96 well microtiter plates were precoated with Polyethylene Immine 0.3% for 1 hour. Wild saxiphilin, prepared from the organism Ethmostigmus rubripes (E.r. SXFN), was incubated for 1 hour at room temperature in the presence of tritiated saxitoxin [³H]STX and several dilutions of both dcSTX hemisuccinate and Biot-link11-STX. Solutions were filtered and saxiphilin was retained on the filters. The signal measured corresponds to the residual radioactivity on the filter and is directly correlated to the concentration of [³H]STX bound to saxiphilin. The data shown indicate that both Biot-link11-STX and dcSTX hemisuccinate were able to compete with [³H]STX for saxiphilin binding. Positive and negative controls were obtained by testing respectively ultrapure water and free non-radioactive saxitoxin.

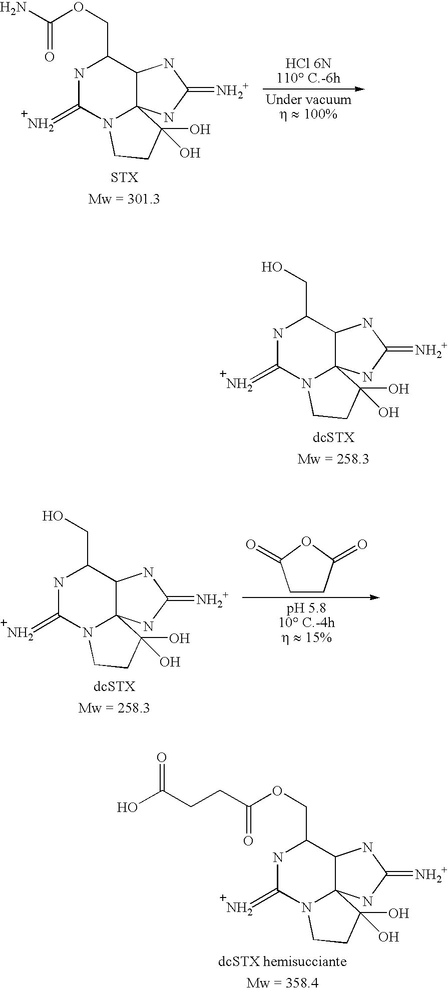

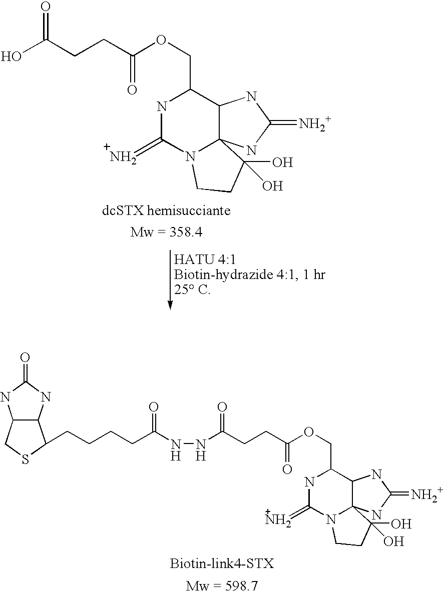

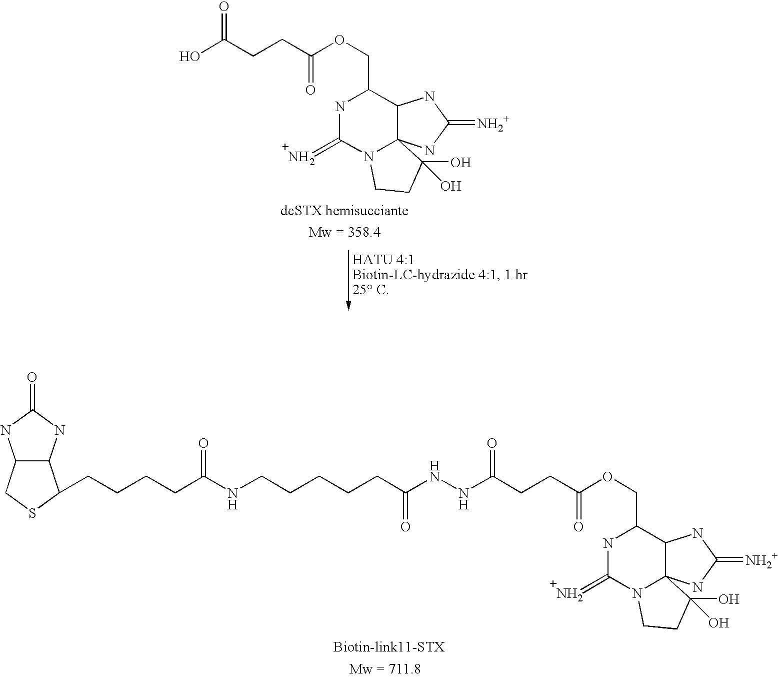

### EXAMPLE 2

Synthesis of Biotin-link18-STX

Saxitoxin isolated from shellfish was converted to decarbamoyl-saxitoxin(dcSTX) by hydrolysis in HCl 6M in a sealed, evacuated glass tube at 110° C. for 4 hours. The solution was freeze dried. The residue was redissolved in 0.05 M acetic acid and the solution passed through a C18 solid phase extraction cartridge. dcSTX was purified by Biogel-P2 chromatography and freeze-dried. The residue was redissolved in anhydrous DMF and reacted overnight at room temperature with excess PMPI (N-(p-Maleimidophenyl) isocyanate) to produce PMPI-STX. PMPI-STX was purified by reversed-phase HPLC-MS. NHS-LC-Biotin was reacted with cysteamine in 10 mM sodium phosphate buffer pH 7.7 at room temperature for 3 min. The final product, a sulfhydryl derivative of Biotin was purified by C18 solid phase extraction and freeze-dried overnight. PMPI-STX was redissolved in 10 mM sodium phosphate buffer pH 6.8 and reacted with an excess of sulfhydryl biotin for 15 min at room temperature producing Biotin-link18-STX, subsequently purified by LC-MS. Biotin-link18-STX was characterized by both reversed-phase and hydrophilic interaction chromatography coupled to electrospray ionisation—tandem mass spectrometry (ESI-MS/MS). The mass spectrum evidenced the presence of a doubly charged ion at m/z=444.2 Da.

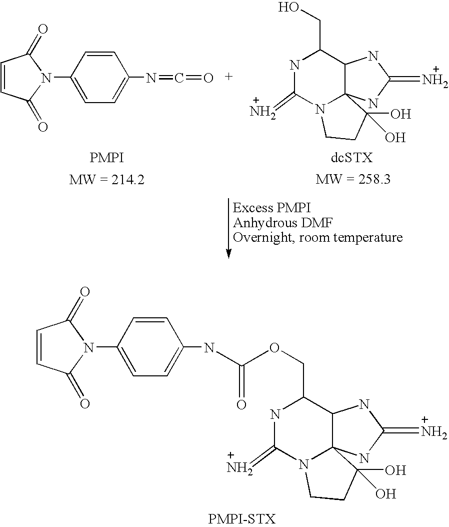

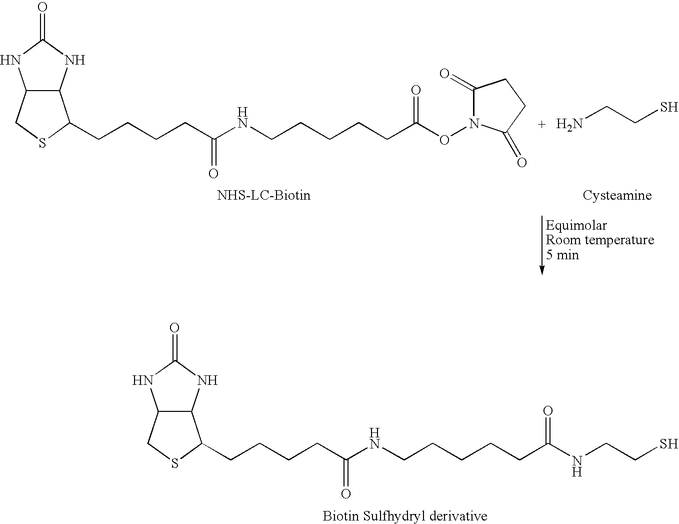

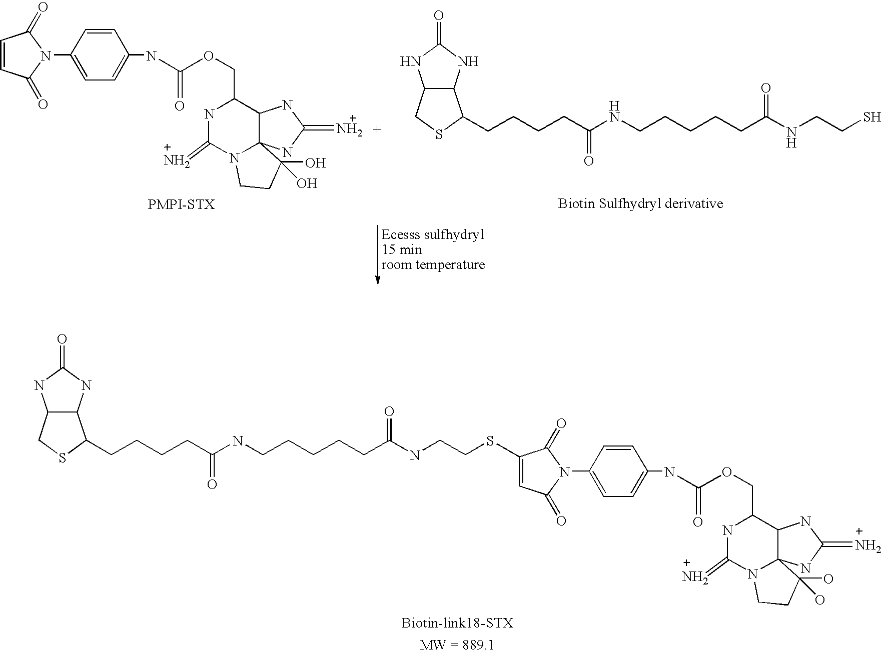

### EXAMPLE 3

Preparation of Avidin.Biotin-link4-STX, Streptavidin.Biotin-link4-STX, Avidin.Biotin-link11-STX and Streptavidin.Biotin-link11-STX Complexes

Streptavidin.Biotin-link4-STX and Streptavidin.Biotin-link11-STX: 324.8 pmol of Biotin-link4-STX and 90.4 pmol Biotin-link11-STX were mixed each with 30 μg Immunopure Streptavidin in 10 mM phosphate buffer (pH 6.5) and incubated for 2 hours at +4° C. 400 μL 0.1% formic acid were added and the solution filtered down to 30 μL using 5,000 cut-off microdialysis centrifuge tubes. The same step was repeated once. Then 200 μL 0.1% formic acid were added and the solution filtered down to 30 μL again. The final volumes were adjusted to 65 μL for Biotin-link4-STX (final concentration of 5 μM) and to 90 μL for Biotin-link11-STX (final concentration of 1 μM) with water.

Avidin.Biotin-link4-STX and Avidin.Biotin-link11-STX: 324.8 pmol of Biotin-link4-STX and 90.4 pmol Biotin-link11-STX were mixed each with 50 μg Immunopure Avidin in 10 mM phosphate buffer (pH 6.5) and incubated for 2 hours at +4° C. 400 μL 0.1% formic acid were added and the solution filtered down to 30 μL using 5,000 cut-off microdialysis centrifuge tubes. The same step was repeated once. Then 200 μL 0.1% formic acid were added and the solution filtered down to 30 μL again. The final volumes were adjusted to 65 μL for Biotin-link4-STX (final concentration of 5 μM) and to 90 μL for Biotin-link11-STX (final concentration of 1 μM) with water.

### EXAMPLE 4

Competition Experiments

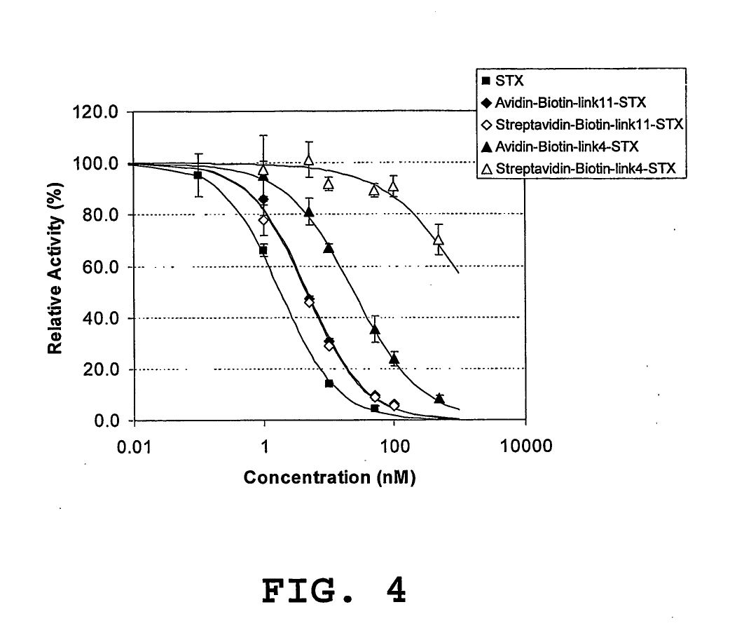

96-well GF/B microtitre filter plates (Millipore) were presoaked with 0.3% (w/v) PEI for at least 1 hour prior to the addition of reagents. All reactions were performed in a total volume of 150 μl containing 20 mM MOPS-NaOH (pH 7.4), 200 mM NaCl, 2 nM [³H]STX and a 70-fold dilution of a crude saxiphilin extract (total protein concentration ˜10 mg/mL). Experiments were allowed to equilibrate for one hour prior to aspiration through the membrane. Wells were rinsed twice with 200 μl of water. Serial dilutions of unlabelled STX, Avidin.Biotin-link4-STX, Streptavidin.Biotin-link4-STX (up to 500 nM), Avidin.Biotin-link11-STX and Streptavidin.Biotin-link11-STX (up to 100 nM) were allowed to compete with [³H]STX 1.8 nM for saxiphilin binding sites (FIG. 4).

Calculation of IC₅₀

Competition curves (FIG. 4.) were fitted using the equation Fraction bound=[STX]ⁿ/([STX]ⁿ+IC₅₀ⁿ), with a Hill slope n=1 (except for Avidin.Biotin-link4-STX where n=0.85 and Streptavidin.Biotin-link4-STX where n=0.7), IC₅₀ being the concentration which caused 50% inhibition, and [STX] the concentration of saxitoxin or saxitoxin analogue.

| | IC₅₀ |
|---|---|
| Compound tested | (nM) |
| Unlabelled STX | 1.6 |
| Avidin.Biotin-link11- | 4.7 |
| STX | |
| Streptavidin.Biotin | 4.5 |
| link11-STX | |
| Avidin.Biotin-link4- | 24 |
| STX | |
| Streptavidin.Biotin- | >1500 |
| link4-STX | |

The use of a linker of more than 4 atoms length between biotin and STX allows the binding of Avidin—Biotin-linker-STX to saxiphilin, although the affinity is significantly improved by extending the linker to 11 atoms (IC₅₀ about five times lower). The effect of the length of the linker is even more visible using Streptavidin instead of Avidin. As a matter of fact, it is well known that the steric hindrance is increased with Streptavidin as a result of the particular conformation of its binding site. Streptavidin.Biotin-link4-STX could not displace radiolabelled STX at concentrations as high as 500 nM, showing a weak affinity for saxiphilin (estimated IC₅₀>1.5 μM). On the contrary, the use of a linker of 11 atoms allows the strong binding of Streptavidin.Biotin-link11-STX to saxiphilin, with a similar IC₅₀ as Avidin.Biotin-link11-STX (IC₅₀=4.5 nM)

### EXAMPLE 5

Detection of Saxitoxin by Surface Plasmon Resonance

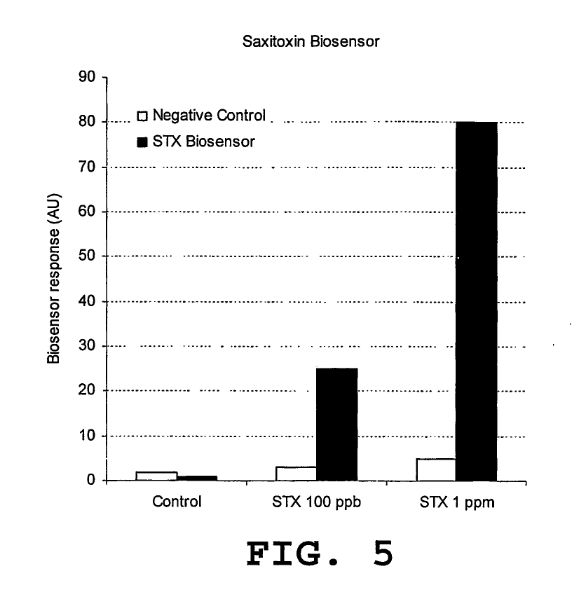

Using a Biacore X system, 140 μL of Biotin-link11-STX were immobilised onto streptavidin-coated membranes (SA chip, Biacore, Uppsala) at a flow rate of 1 μL/min. A reference membrane was obtained by binding pure Biotin-LC-hydrazide to an identical streptavidin membrane on the same chip. A preparation of Ethmostigmus rubripes saxiphilin in physiological buffer pH 7.4 was then injected and washed extensively with buffer. Standard saxitoxin was injected and the absolute displacement of saxiphilin measured by comparison with the reference signal. The histogram on FIG. 5 illustrates the detection of 100 ppb to 1 ppm saxitoxin by surface plasmon resonance using Biotin-link11-STX as the capture probe.

1-33. (canceled)

34. A method for binding a saxiphilin protein obtainable from a source organism and a paralytic shellfish toxin moiety, including the steps of:
(1) providing a paralytic shellfish toxin conjugate comprising the paralytic shellfish toxin moiety bound via a linker comprising 5 atoms or more through a site other than the binding site for saxiphilin directly or indirectly to a biotin moiety;
(2) exposing the paralytic shellfish toxin conjugate to a sample putatively comprising said saxiphilin and to (strept) avidin; and
(3) allowing binding through the paralytic shellfish toxin moiety to the saxiphilin and through the biotin moiety to (strept) avidin;
wherein the paralytic shellfish toxin moiety is characterized by a greater binding affinity for the saxiphilin compared with a binding affinity for the saxiphilin by a paralytic shellfish toxin moiety of a paralytic shellfish toxin conjugate comprising a linker of 4 atoms or less.

35. The method of claim 35 wherein the sample further putatively comprises a paralytic shellfish toxin.

36. The method of claim 35 wherein the method further includes the step of competing binding to the paralytic shellfish toxin moiety by the paralytic shellfish toxin and the saxiphilin.

37. The method of claim 35 wherein the paralytic shellfish toxin comprises saxitoxin.

38. The method of claim 34 wherein the (strept) avidin is immobilized.

39. The method of claim 38 wherein the (strept) avidin is immobilized by a covalent link to a solid support.

40. The method of claim 39 wherein the solid support is a medium for affinity purification.

41. The method of claim 40 wherein the medium is selected from the group consisting of a matrix, beads, magnetic beads, dendrimers, cellulose, polystyrene gel, cross-linked dextran, polyacrylamide gel, porous silica, agarose, sepharose, and a combination thereof.

42. The method of claim 39 wherein the solid support is adapted for use in a biosensing device.

43. The method of claim 42 wherein the solid support is selected from the group consisting of a membrane, microlever, electrode, chemically activated glass surface, and a combination thereof.

44. The method of claim 34 wherein the (strept) avidin carries a detectable label.

45. The method of claim 44 wherein the detectable label is selected from the group consisting of a fluorescent label, radioactive label, chemiluminescent label, colloidal gold, latex bead, liposome, dendrimer, oligonucleotide, peptidonucleic acid, enzyme, and a combination thereof.

46. The method of claim 45 wherein the enzyme is selected from the group consisting of alkaline phosphatase, horseradish peroxidase, betagalactosidase, and a combination thereof.

47. The method of claim 34 wherein the saxiphilin is covalently linked to a solid support.

48. A method as claimed in claim 34 wherein the saxiphilin carries a detectable label.

49. The method of claim 48 wherein the detectable label is selected from the group consisting of a fluorescent label, radioactive label, chemiluminescent label, colloidal gold, latex bead, liposome, dendrimer, oligonucleotide, peptidonucleic acid, enzyme, and a combination thereof.

50. The method of claim 49 wherein the enzyme is selected from the group consisting of alkaline phosphatase, horseradish peroxidase, betagalactosidase, and a combination thereof.

51. The method of claim 34 wherein the paralytic shellfish toxin conjugate comprises a saxitoxin.

52. A method for one or more of detecting, characterizing, isolating, or purifying a saxiphilin protein obtainable from a source organism, comprising:
(1) providing a paralytic shellfish toxin conjugate comprising a paralytic shellfish toxin moiety bound via a linker comprising 5 atoms or more through a site other than the binding site for saxiphilin to a biotin moiety;
(2) exposing the paralytic shellfish toxin conjugate to a sample putatively comprising the saxiphilin to create a reaction mixture and to (strept) avidin; and
(3) allowing binding through the paralytic shellfish toxin moiety to the saxiphilin and through the biotin moiety to (strept) avidin to form a complex; and
(4) detecting, characterizing, isolating and/or purifying the saxiphilin through the complex;
wherein the paralytic shellfish toxin moiety is characterized by a greater binding affinity for the saxiphilin compared with a binding affinity for the saxiphilin by a paralytic shellfish toxin moiety of a paralytic shellfish toxin conjugate comprising a linker of 4 atoms or less.

53. The method of claim 52 wherein the (strept) avidin is immobilized.

54. The method of claim 53 wherein the (strept) avidin is immobilized by a covalent link to a solid support.

55. The method of claim 54 wherein the solid support is a medium for affinity purification.

56. The method of claim 55 wherein the medium is selected from the group consisting of a matrix, beads, magnetic beads, dendrimers, cellulose, polystyrene gel, cross-linked dextran, polyacrylamide gel, porous silica, agarose, sepharose, and a combination thereof.

57. The method of claim 53 wherein the paralytic shellfish toxin conjugate is immobilized to the medium prior to exposure to the sample.

58. The method of claim 54 wherein the paralytic shellfish toxin conjugate is exposed to the sample so as to form a paralytic shellfish toxin complex for capture by the medium.

59. The method of claim 54 further comprising the step of washing the saxiphilin from the sold support to isolate said saxiphilin, to purify said saxiphilin, or a combination thereof.

60. The method of claim 55 further comprising the step of washing the saxiphilin from the medium to isolate said saxiphilin, to purify said saxiphilin, or a combination thereof.

61. The method of claim 54 wherein the solid support is adapted for use in a biosensing device.

62. The method of claim 52 wherein the (strept) avidin carries a detectable label.

63. The method of claim 62 wherein the detectable label is selected from the group consisting of a fluorescent label, radioactive label, chemiluminescent label, colloidal gold, latex bead, liposome, dendrimer, oligonucleotide, peptidonucleic acid, enzyme, and a combination thereof.

64. The method of claim 63 wherein the enzyme is selected from the group consisting of alkaline phosphatase, horseradish peroxidase, betagalactosidase, and a combination thereof.

65. The method of claim 52 wherein the saxiphilin is covalently linked to a solid support.

66. The method of claim 52 wherein the saxiphilin carries a detectable label.

67. The method of claim 66 wherein the detectable label is selected from the group consisting of a fluorescent label, radioactive label, chemiluminescent label, colloidal gold, latex bead, liposome, dendrimer, oligonucleotide, peptidonucleic acid, enzyme, and a combination thereof.

68. The method of claim 67 wherein the enzyme is selected from the group consisting of alkaline phosphatase, horseradish peroxidase, betaglactosidase, and a combination thereof.

69. The method of claim 52 wherein the paralytic shellfish toxin conjugate comprises a saxitoxin.

70. A method for capturing one or both of an antibody to a paralytic shellfish toxin or a RNA aptamer which binds paralytic shellfish toxins, comprising:
(1) providing a paralytic shellfish toxin conjugate comprising a paralytic shellfish toxin moiety bound via a linker comprising 5 atoms or more through a site other than the binding site for saxiphilin to a biotin moiety;
(2) exposing the paralytic shellfish toxin conjugate to a sample putatively comprising said antibody or aptamer and to (strept) avidin; and
(3) allowing binding through the paralytic shellfish toxin moiety to the antibody or aptamer and through the biotin moiety to (strept) avidin;
wherein the paralytic shellfish toxin moiety is characterized by a greater binding affinity for the saxiphilin compared with a binding affinity for the saxiphilin by a paralytic shellfish toxin moiety of a paralytic shellfish toxin conjugate comprising a linker of 4 atoms or less.

71. A biotin/(strep) avidin system including a paralytic shellfish toxin conjugate, said system comprising a paralytic shellfish toxin moiety bound via a linker comprising 5 atoms or more and through a site other than the binding site for saxiphilin to a biotin moiety; wherein the paralytic shellfish toxin moiety is characterized by a greater binding affinity for the saxiphilin compared with a binding affinity for the saxiphilin by a paralytic shellfish toxin moiety of a paralytic shellfish toxin conjugate comprising a linker of 4 atoms or less.

72. The system including a paralytic shellfish toxin conjugate of claim 71 wherein the linker is joined to the paralytic shellfish toxin moiety through C₁₂ or C₁₃ of saxitoxin or the equivalent position in other paralytic shellfish toxins.

73. The system including a paralytic shellfish toxin conjugate of claim 72 wherein the linker joined through C₁₃ of decarbamoylsaxitoxin.

74. The system including a paralytic shellfish toxin conjugate of claim 71 wherein the linker comprises 5 to 20 atoms.

75. The system including a paralytic shellfish toxin conjugate of claim 74 wherein the linker comprises 11 to 18 atoms.

76. A complex comprising the paralytic shellfish toxin conjugate of claim 38 complexed to saxiphilin through the paralytic shellfish toxin moiety.

77. A complex comprising a paralytic shellfish toxin conjugate as claimed in claim 71 complexed to (strept) avidin through the biotin moiety.

78. The complex of claim 77 wherein the (strept) avidin comprises a detectable label.

79. The complex of claim 78 wherein the detectable label is selected from the group consisting of a fluorescent label, radioactive label, chemiluminescent label, colloidal gold, latex bead, liposome, dendrimer, oligonucleotide, peptidonucleic acid, enzyme, and a combination thereof.

80. The complex of claim 79 wherein the enzyme is selected from the group consisting of alkaline phosphatase, horseradish peroxidase, betagalactosidase, and a combination thereof.

81. The complex of claim 77 wherein the (strept) avidin is covalently linked to a solid support.

82. An affinity purification medium comprising the complex of claim 76.

83. The affinity purification medium of claim 82 wherein the medium is selected from the group consisting of a matrix, beads, magnetic beads, dendrimers, cellulose, polystyrene gel, cross-linked dextran, polyacrylamide gel, porous silica, agarose, sepharose, and a combination thereof.

84. A paralytic shellfish toxin biosensing device comprising the complex of claim 76.

85. A paralytic shellfish toxin biosensing device comprising saxiphilin covalently linked to a solid support and means for detecting binding of the paralytic shellfish toxin conjugate of claim 71 thereto.

86. The paralytic shellfish toxin biosensing device of claim 85 where the paralytic shellfish toxin conjugate is bound to (strept) avidin.

87. The paralytic shellfish toxin biosensing device as claimed in claim 86 where an increase in mass is detected.
